(19) United States
(12) Patent Application Publication
(10) Pub. No.: US 2012/0301554 A1
(43) Pub. Date: Nov. 29, 2012

(54) EFFECT PIGMENTS

(75) Inventor: Helge Bettina Kniess, Rossdorf (DE)

(73) Assignee: MERCK PATENT GESELLSCHAFT MIT BESCHRANKTER HAFTUNG, Darmstadt (DE)

(21) Appl. No.: 13/521,890

(22) PCT Filed: Dec. 17, 2010
(86) PCT No.: PCT/EP10/07742

§ 371 Date: Jul. 12, 2012

(30) Foreign Application Priority Data
Jan. 15, 2010 (EP) 10 000 338.3

(51) Int. Cl. C09D 17/00; C03C 14/00; C08K 3/22; C09D 11/02; A61K 8/19; B82Y 30/00

(52) U.S. Cl. 424/635; 106/456; 106/287.17; 106/439; 106/459; 106/31.13; 501/32; 524/413; 977/755

(57) ABSTRACT

The present invention relates to effect pigments having a substrate which has a coating comprising a complex metal oxide containing copper, iron and manganese, and to a process for the preparation of the effect pigments, to the use of the effect pigments in paints, coatings, powder coatings, printing inks, plastics, ceramic materials, glasses, in cosmetic formulations, for laser marking and for the preparation of pigment preparations and dry preparations.

The present invention relates to effect pigments having a substrate which has a coating comprising a complex metal oxide containing copper, iron and manganese, and to a process for the preparation of the effect pigments, to the use of the effect pigments in paints, coatings, powder coatings, printing inks, plastics, ceramic materials, glasses, in cosmetic formulations, for laser marking and for the preparation of pigment preparations and dry preparations.

Lustre or effect pigments, as are known, for example, from DE 2522572, are employed in many areas of industry, in particular in the area of automotive paints, decorative coating, in plastic, in paints, printing inks and in cosmetic formulations.

Effect pigments having a dark-grey colour are known, for example, from WO 9319131, DE 19522267, EP 0735115 and EP 0601761. A disadvantage of these pigments is, however, that they always have a somewhat bluish-grey colour owing to the content of reduced titanium dioxide (sub-oxide).

Furthermore, grey effect pigments are known whose coloration is generated by the application of CuₓMn₃₋ₓO₄ (x=1.4 or 1.5) to the surface of a titanium dioxide-coated flake-form substrate (EP 0719843). These pigments are likewise not neutral-black or grey, but instead always have a bluish to greenish coloration.

U.S. Pat. No. 2,811,463 describes the preparation of a black, substrate-free pigment consisting of manganese oxide, copper oxide and iron oxide as calcination product. However, this pigment has no lustre at all.

Black effect pigments are in many cases based on carbon. Pigments of this type are known from DE 4227082 A1, DE 3636076 A1, DE 3617430 A1 and EP 0246523. Black lustre pigments are prepared either through the use of carbon black, by decomposition processes of organic compounds or by temperature-dependent calcination of hydrocarbons. EP 1520883 discloses black, lustrous interference pigment having an adjustable colour content, i.e. having a gold, green, red or blue tint.

The black pigments known from the prior art have the disadvantage that they have a dull lustre and a grey-black or brown-black colour or exhibit interference colours which are very highly dependent on the viewing angle, which is undesired in the majority of applications. Furthermore, these pigments are in some cases very difficult to prepare or reproduce.

The object of the present invention is therefore to prepare a lustrous effect pigment having a neutral-grey or neutral-grey to -black mass tone.

Surprisingly, novel, colour-neutral, lustrous effect pigments have now been found which are at the same time distinguished by high hiding power and good processability, which do not have pronounced goniochromaticity, or have none at all, and which are easy to prepare. The invention therefore relates to an effect pigment having a substrate which has a coating comprising a complex metal oxide containing copper, iron and manganese.

The coating which is essential to the invention preferably comprises complex metal oxides comprising single-phase mixed crystals of copper oxide, iron oxide and manganese oxide. The complex metal oxide preferably exhibits a spinel structure. In the spinel structure, at least two different types of cation (divalent and tri- or tetravalent cations) occupy different types of lattice sites, namely eight point positions with tetrahedral coordination and 16 point positions with octahedral coordination per elemental cell. In the case of normal spinels, all 16 tri- or tetravalent cations are located in the octahedral lattice sites, in the case of inverse spinels, eight tri- or tetravalent cations and eight divalent cations have octahedral coordination. Owing to certain degrees of freedom, the cation distributions in spinels frequently have ordered/unordered states.

Iron and manganese can be in both divalent and trivalent form in the complex metal oxide. The complex metal oxide may additionally contain further tri- and/or divalent cations, preferably aluminium, cobalt and/or titanium cations. Preferred amounts of aluminium oxide, cobalt oxide and/or titanium oxide here are 0-10% by weight, based on the complex metal oxide.

The complex metal oxide preferably conforms to the formula CuₓFe_{y}Mn_{3-xy}O₄, where x=1-1.4 and y=0.1-1. The complex metal oxides are particularly preferably stoichiometric spinels. A copper spinel of the form Cu(Fe,Mn)₂O₄ is particularly preferred.

The amount of complex metal oxide is 2.5-60% by weight, preferably 20-60% by weight, in particular 20-50% by weight, based on the substrate. In a preferred variant, the coating which is essential to the invention may partially comprise titanium dioxide in addition to the complex metal oxide. The thickness of the coating comprising a complex metal oxide which is essential to the invention is preferably 1 to 350 nm, in particular 10 nm to 300 nm and very particularly preferably 20 to 200 nm.

Suitable substrates for the effect pigments according to the invention are, for example, all known flake-form support materials, preferably transparent or semitransparent flakes. Preference is given to flake-form support materials coated with one or more high- or low-refractive-index, transparent or semitransparent metal-oxide layers. Titanium dioxide-coated mica is particularly preferred.

Suitable support materials are, for example, phyllosilicates, in particular synthetic or natural mica, glass flakes, metal flakes, SiOₓ flakes (x=≦2.0, preferably x=2), Al₂O₃ flakes, TiO₂ flakes, synthetic or natural iron-oxide flakes, graphite flakes, structured pigments, synthetic support-free flakes, liquid crystal polymers (LCPs), holographic pigments, BiOCl flakes or mixtures of the said flakes. Preference is given to flakes comprising synthetic or natural mica, glass flakes, SiO0₂ flakes and Al₂O₃ flakes, in particular mica flakes.

In general, the flake-form support materials have a thickness between 0.05 and 5 μm, in particular between 0.1 and 4.5 μm. Glass flakes preferably have a thickness of ≦1 μm, in particular ≦900 nm and very particularly preferably ≦500 nm. The size of the support materials is not crucial per se and can be matched to the particular application. The particle size is usually 1-350 μm, preferably 2-200 pm and in particular between 5-150 μm. In general, both coarse flakes having particle sizes of 10-200 μm, preferably 40-200 μm, in particular 10-130 μm, and also fine flakes having particle sizes of 1-60 μm, preferably 5-60 μm, in particular 10-40 μm, can be used. Mixtures consist of flakes having different particle sizes can preferably also be employed.

The particle sizes are determined by means of laser diffraction on the powder or on pigment suspensions using commercially available instruments which are known to the person skilled in the art (for example from Malvern, Horiba). The substrates preferably have a form factor (aspect ratio: diameter/thickness ratio) of 5-750, in particular 10-300 and very particularly preferably 20-200. In addition, the use of other substrates, such as, for example, spherical particles or needle-shaped substrates, which may be covered with the above-mentioned layers, is also possible.

In a preferred embodiment, the support material can be coated with one or more transparent, semitransparent and/or opaque layers comprising metal oxides, metal oxide hydrates, metal suboxides, metals, metal fluorides, metal nitrides, metal oxynitrides or mixtures of these materials. The metal oxide, metal oxide hydrate, metal suboxide, metal, metal fluoride, metal nitride, metal oxynitride layers or the mixtures thereof can have a low refractive index (refractive index <1.8) or a high refractive index (refractive index ≧1.8, preferably ≧2.0.). Suitable metal oxides and metal oxide hydrates are all metal oxides or metal oxide hydrates known to the person skilled in the art, such as, for example, aluminium oxide, aluminium oxide hydrate, silicon oxide, silicon oxide hydrate, iron oxide, tin oxide, cerium oxide, zinc oxide, zirconium oxide, chromium oxide, titanium oxide, in particular titanium dioxide, in the rutile or anatase modification, titanium oxide hydrate and mixtures thereof, such as, for example, ilmenite or pseudo-brookite. Metal suboxides which can be employed are, for example, the titanium suboxides. Suitable metals are, for example, chromium, aluminium, nickel, silver, gold, titanium, copper or alloys, a suitable metal fluoride is, for example, magnesium fluoride. Metal nitrides or metal oxynitrides which can be employed are, for example, the nitrides or oxynitrides of the metals titanium, zirconium and/or tantalum. Metal oxide, metal, metal fluoride and/or metal oxide hydrate layers and very particularly preferably metal oxide and/or metal oxide hydrate layers are preferably applied to the support. Particular preference is given to oxides and/or oxide hydrates of aluminium, silicon, iron, tin and titanium, in particular titanium dioxide, in the rutile or anatase modification, and mixtures of these compounds. Multilayered structures comprising high- and low-refractive-index metal oxide, metal oxide hydrate, metal or metal fluoride layers may furthermore also be present, where high- and low-refractive-index layers preferably alternate. Particular preference is given to layer packages comprising a high-refractive-index layer and a low-refractive-index layer, where one or more of these layer packages may be applied to the support. The sequence of the high- and low-refractive-index layers can be matched to the support here in order to incorporate the support into the multilayered structure.

Suitable substrates are, in particular, flake-form support materials which have been coated with one or more high- or low-refractive-index, transparent or semitransparent metal-oxide layers. Preference is given to supports coated with one or more metal-oxide layers. Particular preference is given to titanium dioxide-coated mica or mica which is mono- or multicoated with TiO₂/SnO₂.

The metal oxide, hydroxide and/or oxide hydrate layers are preferably applied by wet-chemical methods. Methods of this type are known to the person skilled in the art and, for example, in DE 25 22 572. Examples and embodiments of the above-mentioned materials and pigment structures are also found, for example, in Research Disclosures RD 471001 and RD 472005. In the case of wet coating, the substrate is suspended in water, and one or more hydrolysable metal salts are added at a pH which is suitable for hydrolysis, which is selected so that the metal oxides or metal oxide hydrates are precipitated directly onto the flakes without secondary precipitations occurring. The pH is usually kept constant by simultaneous metered addition of a base or acid. If desired, the pigments can be separated off, dried and optionally calcined after application of individual coatings and then re-suspended for the precipitation of the further layers. Furthermore, the coating may also be carried out in a fluidised-bed reactor by gas-phase coating.

The coatings on the support material preferably consist of simple or complex metal oxides, metals, nitrides or oxynitrides, such as, for example, TiO₂, ZrO₂, ZnO, SnO₂, SiO₂, SiO(OH)₂, Al₂O₃, AlO(OH), B₂O₃ or mixtures thereof or also BiOCl or also MgF₂. TiO₂ is particularly preferred.

The thickness of the metal oxide, metal oxide hydrate, metal suboxide, metal, metal fluoride, metal nitride, metal oxynitride layers or a mixture thereof is usually 1 to 1000 nm, preferably 1 to 800 nm, in particular 1 to 600 nm. Layer thicknesses of 1 to 300 nm, in particular 1 to 100 nm, are particularly suitable. The thickness of the metal layers is preferably 4 to 60 nm.

The coating comprising a complex metal oxide which is essential to the invention may be present as outer oxide coating or coated with with one or more high- or low-refractive-index, transparent or semitransparent metal-oxide layers, preferably with a TiO₂ layer. The coating comprising a complex metal oxide is preferably on the outside.

The coating comprising a complex metal oxide which is essential to the invention is produced by forming the complex metal oxide on a substrate by adding water-soluble copper, iron and manganese salts to an aqueous suspension of the substrate in such a way that the water-containing oxides are generated on the substrate, optionally partially mixed with the titanium dioxide layer, where the water-soluble copper, iron and manganese salts may be metered in simultaneously or successively.

It is preferred for the manganese salt solution to be added first, optionally at the same time as the titanium salt solution, and then for the copper salt solution and subsequently the iron salt solution to be metered in. Suitable metal salts are, in particular, halides, nitrates and sulfates, in particular chlorides and sulfates. The precipitation of the metal oxides is achieved through suitable pH and temperature conditions. The amounts and concentrations to be employed which are necessary for a desired pigment composition can be determined routinely by the person skilled in the art.

The effect pigments of the invention may also have one or more layers comprising high- and/or low-refractive-index, transparent or semitransparent metal compounds, in particular comprising TiO₂, ZrO₂, ZnO, SnO₂, SiO₂, SiO(OH)₂, Al₂O₃, AlO(OH), B₂O₃ or mixtures thereof, above the coating comprising a complex metal oxide.

The pigment prepared by the process described is separated off, washed, dried, preferably at 80-150° C., and calcined in air for 30-60 minutes at 400-850° C., preferably 500-700° C., resulting in the formation of the complex metal oxide.

A further process for the preparation of effect pigments having a coating according to the invention comprising a complex metal oxide comprises the calcination of a mixture of a) TiO₂/CuO-coated mica, b) TiO₂/FeO-coated mica and c) TiO₂/MnO-coated mica. The coated micas are coated separately by the processes described above, washed and dried. The mixture is then prepared, and the micas coated in this way are calcined jointly in air for 30-60 minutes at 400-850° C., preferably 500-700° C., resulting in the formation of the complex metal oxide.

In order to increase the light, water and weather stability, it is frequently advisable, depending on the area of application, to subject the finished pigment to a post-coating or post-treatment. Suitable post-coatings or post-treatments are all post-coatings known to the person skilled in the art. This post-coating further increases the chemical stability or simplifies handling of the pigment, in particular incorporation into various media. In order to improve the wettability, dispersibility and/or compatibility with the user media, further conventional functional coatings, for example with silanes, can be applied.

Particular preference is given to an effect pigment consisting of, in this sequence, a mica substrate, optionally a tin dioxide coating, a titanium dioxide coating, a coating comprising a complex metal oxide having a spinel structure of the form Cu(Fe,Mn)₂O₄, where the titanium dioxide coating and the coating comprising a complex metal oxide may be partially or fully, preferably partially, mixed, and optionally a post-coating.

For the various applications, the effect pigments according to the invention can also advantageously be used as a blend with fillers, organic dyes and/or pigments, such as, for example, transparent and opaque white, coloured and black pigments, and with flake-form iron oxides, organic pigments, holographic pigments, LCPs (liquid crystal polymers), interference pigments and conventional transparent, coloured and black lustre pigments based on metal oxide-coated flakes based on mica, glass, Al₂O₃, Fe₂O₃, SiO₂, etc. The effect pigments according to the invention can be mixed in any ratio with commercially available pigments and fillers.

Fillers which may be mentioned are, for example, natural and synthetic mica, nylon powder, pure or filled melanin resins, talc, glasses, kaolin, oxides or hydroxides of aluminium, magnesium, calcium, zinc, BiOCl, barium sulfate, calcium sulfate, calcium carbonate, magnesium carbonate, carbon, and physical or chemical combinations of these substances. There are no restrictions regarding the particle shape of the filler. It can be, for example, flake-form, spherical or needle-shaped in accordance with requirements.

The effect pigments according to the invention can be used in paints (automotive and industrial coatings, solvent- and water-based, powder coatings), plastics, printing inks, ceramic glazes or cosmetic formulations. They can also be used in the form of preparations (pearlets, pastes), for example for use in printing inks or plastics.

The effect pigments according to the invention are furthermore suitable for the preparation of flowable pigment preparations and dry preparations comprising one or more effect pigments according to the invention, binders and optionally one or more additives. Dry preparations are also taken to mean preparations which comprise 0 to 8% by weight, preferably 2 to 8% by weight, in particular 3 to 6% by weight, of water and/or a solvent or solvent mixture. The dry preparations are preferably in the form of pellets, granules, chips, sausages or briquettes and have particle sizes of 0.2-80 mm. The dry preparations are used, in particular, in the preparation of printing inks and in cosmetic formulations.

The effect pigments according to the invention are compatible with a multiplicity of colour systems, preferably from the area of paints, coatings and printing inks. For the preparation of printing inks, a multiplicity of binders, in particular water-soluble types, is suitable, as marketed, for example, by the companies BASF, Marabu, Pröll, Sericol, Hartmann, Gebr. Schmidt, Sicpa, Aarberg, Siegberg, GSB-Wahl, Follmann, Ruco or Coates Screen INKS GmbH. The printing inks can be built up on the basis of water or on the basis of a solvent. The pigments are furthermore also suitable for the laser marking of paper and plastics, and for applications in the agricultural sector, for example for greenhouse sheeting, and, for example, for the colouring of tarpaulins.

The invention furthermore also relates to the use of the effect pigments in formulations, such as paints, printing inks, security printing inks, coatings, powder coatings, plastics, ceramic materials, glasses, in cosmetic formulations, as dopant for the laser marking of papers and plastics and for the preparation of pigment preparations and dry preparations.

The disclosures in the references cited hereby also expressly belong to the disclosure content of the present application. The following examples explain the present invention in greater detail without restricting the scope of protection.

### EXAMPLES

### Example 1

### Preparation of A Lustrous, Semitransparent Effect Pigment Having A Dark-Grey Mass Tone And Having The Composition Mica, Tin Dioxide, Titanium Dioxide, Cu(Fe,Mn)₂O₄

100 g of mica having a particle size of 10-60 μm are heated to 75° C. with stirring in 1.5 l of demineralised water. The pH of the suspension is then adjusted to 1.8 using a 5% hydrochloric acid. This is followed by the metered addition of a tin tetrachloride solution (comprising 4.5 g of a 50% SnCl₄ solution and 12 g of concentrated hydrochloric acid in 50 g of deionised water), where the pH is kept constant by simultaneous dropwise addition of a 20% sodium hydroxide solution. When the addition is complete, the mixture is stirred for a further 15 min. This is followed by the metered addition of a titanium tetrachloride/manganese dichloride solution (34.8 g of MnCl₂*2 H₂O dissolved in 250 ml of a TiCl₄ solution having a content of 340 g of TiCl₄/l), where the pH is kept constant by simultaneous dropwise addition of a 32% sodium hydroxide solution. When the addition is complete, the mixture is stirred for a further 15 min. The copper chloride solution (29 g of CuCl₂*2 H₂O dissolved in 150 ml of deionised water) and subsequently the iron sulfate solution (36.8 g of FeSO₄*7 H₂O dissolved in 100 ml of deionised water) is then metered in.

When the addition is complete, the mixture is stirred for a further 15 min. The pH is subsequently adjusted slowly to 9.8 by addition of 20% sodium hydroxide solution, and the mixture is stirred for a further 30 min. The product is filtered off, washed, dried, calcined at 600-700° C. and sieved through a 100 μm sieve, giving a pigment having a silvery lustre and a colour-neutral dark-grey mass tone and high hiding power.

Paint cards are subsequently prepared from the effect pigment after incorporation into nitrocellulose lacquer and these are measured colouristically.

Chroma black card: 7.42

Chroma white card: 1.58

### Comparative Example

Preparation of a lustrous, semitransparent effect pigment having a dark-grey mass tone and having the composition mica, tin dioxide, titanium dioxide, CuₓMn₃₋ₓO₄ In Accordance With EP 0719843, Where x=1.4

100 g of mica having a particle size of 10-60 μm are heated to 75° C. with stirring in 1.5 l of demineralised water. The pH of the suspension is then adjusted to 1.8 using a 5% hydrochloric acid. This is followed by the metered addition of a tin tetrachloride solution (comprising 4.5 g of a 50% SnCl₄ solution and 12 g of concentrated hydrochloric acid in 50 g of deionised water), where the pH is kept constant by simultaneous dropwise addition of a 20% sodium hydroxide solution. When the addition is complete, the mixture is stirred for a further 15 min. This is followed by the metered addition of a titanium tetrachloride/manganese dichloride solution (34.8 g of MnCl₂*2 H₂O dissolved in 250 ml of a TiCl₄ solution having a content of 340 g of TiCl₄/l), where the pH is kept constant by simultaneous dropwise addition of a 32% sodium hydroxide solution. When the addition is complete, the mixture is stirred for a further 15 min. The copper chloride solution (30.0 g of CuCl₂*2 H₂O dissolved in 150 ml of deionised water) is then metered in. When the addition is complete, the mixture is stirred for a further 15 min. The pH is subsequently adjusted slowly to 9.8 by addition of 20% sodium hydroxide solution, and the mixture is stirred for a further 30 min. The product is filtered off, washed, dried, calcined at 600-700° C. and sieved through a 100 μm sieve, giving an effect pigment having a silvery lustre and a blue-grey mass tone and high hiding power.

Paint cards are subsequently prepared from the pigment after incorporation into nitrocellulose lacquer and these are measured colouristically.

Chroma black card: 13.30

Chroma white card: 1.81

1. Effect pigment having a substrate which has a coating comprising a complex metal oxide containing copper, iron and manganese.

2. Effect pigment according to claim 1, characterised in that the complex metal oxide has a spinel structure.

3. Effect pigment according to one of claim 1, characterised in that the amount of complex metal oxide is 2.5-60% by weight, in particular 20-50% by weight, based on the substrate.

4. Effect pigment according to claim 1, characterised in that the complex metal oxide conforms to the formula CuₓFe_{y}Mn_{3-x-y}O₄, where x=1-1.4 and y=0.1-1.

5. Effect pigment according to claim 1, characterised in that the complex metal oxide additionally contains aluminium cations, cobalt cations and/or titanium cations.

6. Effect pigment according to claim 1, characterised in that the complex metal oxide is a copper spinel of the form Cu(Fe,Mn)₂O₄.

7. Effect pigment according to claim 1, characterised in that the coating partially comprises titanium dioxide.

8. Effect pigment according to claim 1, characterised in that the coating has a thickness of 1 to 350 nm, preferably 100 to 250 nm.

9. Effect pigment according to claim 1, characterised in that the substrate is in flake form.

10. Effect pigment according to claim 1, characterised in that the substrate is natural or synthetic mica, BiOCl flakes, glass flakes, Fe₂O₃ flakes, graphite flakes, Al₂O₃ flakes, SiO₂ flakes or TiO₂ flakes or mixtures of these flakes, in particular natural or synthetic mica.

11. Effect pigment according to claim 1, characterised in that the substrate has one or more layers comprising high-and/or low-refractive-index, transparent or semitransparent metal compounds beneath or above the coating comprising a complex metal oxide.

12. Effect pigment according to claim 11, characterised in that the metal compounds are selected from TiO₂, ZrO₂, ZnO, SnO₂, SiO₂, SiO(OH)₂, Al₂O₃, AlO(OH), B₂O₃ and mixtures thereof.

13. Effect pigment according to claim 1, characterised in that it has an inorganic or organic post-coating.

14. Process for the preparation of the effect pigments according to claim 1, characterised in that the coating of the flake-form substrates is carried out by wet-chemical methods, by CVD methods or PVD methods.

15. A composition comprising an effect pigment according to claim 1 and at least one further component used for paints, coatings, powder coatings, printing inks, plastics, ceramic materials, glasses, in cosmetic formulations, for laser marking and for the preparation of pigment preparations and dry preparations.
